(19) United States
(12) Patent Application Publication
(10) Pub. No.: US 2026/0013963 A1
(43) Pub. Date: Jan. 15, 2026

(54) ANTERIOR HIP DRAPE AND METHODS OF USE

(71) Applicant: KPR U.S., LLC, Mansfield, MA (US)

(72) Inventors: Despina TOMBOULIDES, Boston, MA (US); Regina GOODWIN, El Paso, TX (US); Kerry H. BEAVEN, Carver, MA (US); Jennifer A. NADAR, Taunton, MA (US)

(21) Appl. No.: 19/265,363

(22) Filed: Jul. 10, 2025

Related U.S. Application Data
(60) Provisional application No. 63/669,592, filed on Jul. 10, 2024.

(51) Int. Cl. A61B 46/23

(52) U.S. Cl. CPC A61B 46/23

(57) ABSTRACT

A surgical ambipedal drape with a main body portion with a fenestration for accessing a surgical site, the main body portion including: a main body first side portion, a main body second side portion, a main body third side portion, and a main body fourth side portion. The surgical drape further includes a first anesthesia screen extending from the third side portion and a second anesthesia screen extending from the fourth side portion.

### CROSS REFERENCE TO RELATED APPLICATION

This application claims priority to U.S. Provisional Application No. 63/669,592 titled: “ANTERIOR HIP DRAPE AND METHODS OF USE,” filed on Jul. 10, 2025. The entirety of the aforementioned application is incorporated by reference herein.

### TECHNICAL FIELD

This disclosure is related to drape system and methods for using and making a drape system.

### BACKGROUND

The direct anterior approach to the hip for total joint arthroplasty (i.e., hip replacement) has been suggested to have several advantages compared to other popular approaches. Recent emphasis on tissue sparing and minimally invasive outpatient joint replacements has given rise to a significant increase in the utilization the anterior approach for several surgical procedures. Proponents of the anterior approach cite improved recovery times, lower pain levels, improved patient satisfaction as well as improved accuracy on both implant placement/alignment and leg length restoration. Thus, the need exists for a drape and drape system that is usable for anterior approach surgery.

### SUMMARY

The following presents a simplified summary of one or more aspects of the technology disclosed herein in order to provide a basic understanding of such aspects. This summary is not an extensive overview of all contemplated aspects and is intended to neither identify key or critical elements of all aspects nor delineate the scope of any or all aspects. Its sole purpose is to present some concepts of one or more aspects in a simplified form as a prelude to the more detailed description that is presented later.

The technology described herein relates to a surgical drape including: a main body portion with a fenestration for accessing a surgical site, the main body portion including: a main body first side portion; a main body second side portion opposite the first side portion; a main body third side portion extending in a direction substantially perpendicular to the main body first side portion and the main body second side portion; a main body fourth side portion opposite the main body third side portion and extending in a direction substantially perpendicular to the main body first side portion and the main body second side portion; the surgical drape further including: a first anesthesia screen extending away from the third side portion; and a second anesthesia screen extending away from the fourth side portion.

The technology described further relates to a surgical drape, wherein the fenestration is position on the main body portion closer to the first side portion than the second side portion. The technology described further relates to surgical drape including a reinforcement portion at least partially surrounding the fenestration.

The technology described further relates to including at least one pocket for retaining one or more surgical instruments. The technology described further relates including a second pocket for retaining one or more surgical instruments.

The technology described further relates to a surgical drape, wherein the pocket is adhesively adhered to the main body portion.

The technology described further relates to including one or more pockets that have an adhesive backing and are configured to be adhered to the drape.

The technology described further relates to a surgical drape, wherein the main body portion, the first anesthesia screen, and the second anesthesia screen are symmetrical about an axis that bisects the main body portion.

The technology described further relates to a surgical drape, wherein the main body portion is formed of a non-woven material and the first anesthesia screen, and the second anesthesia screen are formed of a transparent material.

The technology described further relates to including a first armboard cover portion and a second armboard cover portion connected to the first anesthesia screen and a third armboard cover portion and fourth armboard cover portion connected to the second anesthesia screen.

The technology described further relates to a surgical drape, wherein the first armboard cover portion, second armboard cover portion, third armboard cover portion, and fourth armboard cover portion are connected to the main body portion.

The technology described further relates to surgical drape, wherein the main body portion further includes: a first outer main body portion extending toward the third side portion; and a second outer main body portion extending toward the fourth side portion, wherein the first outer main body portion is connected to the first armboard cover portion and the second armboard cover portion and the second outer main body portion is connected to the third armboard cover portion, and fourth armboard cover portion.

In some aspects, the techniques described herein relate to a method of forming a surgical drape, comprising: assembling a main body portion with a fenestration for accessing a surgical site, the main body portion including: a main body first side portion; a main body second side portion opposite the main body first side portion; a main body third side portion; a main body fourth side portion opposite the main body third side portion; connecting a first anesthesia drape portion to extend from the third side portion; and a second anesthesia drape portion to extend from the fourth side portion.

In some aspects of the disclosure, the techniques described herein relate to the aforementioned method, wherein the first anesthesia drape portion and the second anesthesia drape portion are formed of a transparent material.

In some aspects of the disclosure, the techniques described herein relate to any of the aforementioned methods, wherein the fenestration is formed closer to the first side portion than the second side portion.

In some aspects of the disclosure, the techniques described herein relate to any of the aforementioned methods, further including providing a reinforcement portion at least partially surrounding the fenestration.

In some aspects of the disclosure, the techniques described herein relate to any of the aforementioned methods, further including connecting a first outer body portion of the main body portion to the first anesthesia drape portion and connecting a second outer body portion to the second anesthesia drape portion.

The disclosure further includes technology related to a surgical drape system including: a package containing an adhesive backed pocket and a surgical drape, the surgical drape including: a main body portion with a fenestration for accessing a surgical site, the main body portion including: a main body first side portion; a main body second side portion opposite the first side portion; a main body third side portion extending in an direction substantially perpendicular to the main body first side portion and the main body second side portion; a main body fourth side portion opposite the main body third side portion and extending in a direction substantially perpendicular to the main body first side portion and the main body second side portion; the surgical drape further including: a first anesthesia screen extending away from the third side portion; and a second anesthesia screen extending away from the fourth side portion. The surgical drape system may further include an adhesive portion, surrounding the fenestration.

To the accomplishment of the foregoing and related ends, the one or more aspects of the disclosure comprise the features hereinafter fully described and particularly pointed out in the claims. The following description and the annexed drawings set forth in detail include certain illustrative features of the one or more aspects. These features are indicative, however, of but a few of the various ways in which the principles of various aspects can be employed, and this description is intended to include all such aspects and their equivalents.

### BRIEF DESCRIPTION OF THE DRAWINGS

Features characteristic of aspects if the technology described herein are set forth as follows, in the appended claims, and in the drawings. In the descriptions that follow, like parts are marked throughout the specification and drawings with the same numerals, respectively. The drawing figures are not necessarily drawn to scale and certain figures can be shown in exaggerated or generalized form in the interest of clarity and conciseness. The disclosure itself, however, as well as a preferred mode of use, further objects and advances thereof, will be best understood by reference to the following detailed description of illustrative aspects when read in conjunction with the accompanying drawings.

FIG. 1A shows one example of a surgical drape according to aspects of the disclosure in an example in-use orientation.

FIG. 1B shows another example of a surgical drape according to aspects of the disclosure in an example in-use orientation.

FIG. 2 shows an example of a surgical drape according to aspects of the disclosure in a first example in-use orientation.

FIG. 3 shows an example of a surgical drape according to aspects of the disclosure in a second example in-use orientation.

FIG. 4 shows a schematic diagram of an example of a surgical drape according to aspects of the disclosure.

FIG. 5A shows a schematic diagram of an example of a surgical drape according to aspects of the disclosure.

FIG. 5B shows a schematic diagram of another example of a surgical drape according to aspects of the disclosure.

FIG. 6 shows an example of a folded drape according to aspects of the disclosure.

FIG. 7 shows examples of a first unfolded orientation and a second unfolded orientation of the example folded drape of FIG. 6 according to aspects of the disclosure.

FIGS. 8A-8C show a flow diagram and example steps of manufacturing a drape according to aspects of the disclosure.

### DETAILED DESCRIPTION

The detailed description set forth below in connection with the appended drawings is intended as a description of various configurations and is not intended to represent the only configurations in which the concepts described herein can be practiced. The detailed description includes specific details for the purpose of providing a thorough understanding of various concepts. However, it will be apparent to those skilled in the art that these concepts can be practiced without these specific details. In some instances, well known structures and components are shown in block diagram form in order to avoid obscuring such concepts.

### I. Terminology

Reference throughout this specification to one aspect, an aspect, one example or an example means that a particular feature, structure or characteristic described in connection with the embodiment or example may be a feature included in at least example of the present disclosure. Thus, appearances of the phrases in one aspect, in an aspect, one example or an example in various places throughout this specification are not necessarily all referring to the same example. Furthermore, the particular features, structures or characteristics may be combined in any suitable combinations and/or subcombinations in one or more embodiments or examples.

Throughout the disclosure, the terms substantially or approximately may be used as a modifier for a geometric relationship between elements or for the shape of an element or component. While the terms substantially or approximately are not limited to a specific variation and may cover any variation that is understood by one of ordinary skill in the art to be an acceptable level of variation, some examples are provided as follows. In one example, the term substantially or approximately may include a variation of less than 10% of the dimension of the object or component. In another example, the term substantially or approximately may include a variation of less than 5% of the object or component. If the term substantially or approximately is used to define the angular relationship of one element to another element, one non-limiting example of the term substantially or approximately may include a variation of 5 degrees or less. These examples are not intended to be limiting and may be increased or decreased based on the understanding of acceptable limits to one of skill in the relevant art.

For purposes of the disclosure, directional terms are expressed generally with relation to a standard frame of reference when the aspects or articles described herein are in an in-use orientation. In some examples, the directional terms are expressed generally with relation to a left-hand coordinate system.

Terms such as a, an, and the, are not intended to refer to only a singular entity, but also include the general class of which a specific example may be used for illustration. The terms a, an, and the, may be used interchangeably with the term at least one. The phrases at least one of and comprises at least one of followed by a list refers to any one of the items in the list and any combination of two or more items in the list. All numerical ranges are inclusive of their endpoints and non-integer values between the endpoints unless otherwise stated.

The terms first, second, third, and fourth, among other numeric values, may be used in this disclosure. It will be understood that, unless otherwise noted, those terms are used in their relative sense only. In particular, certain components may be present in interchangeable and/or identical multiples (e.g., pairs). For these components, the designation of first, second, third, and/or fourth may be applied to the components merely as a matter of convenience in the description.

The terms disposed on, disposed along, disposed with, or disposed toward and variations thereof may define one element can be integral with another element, or that one element can be a separate structure bonded to or placed with or placed near another element.

The term at, for example when referring to something being located at a specific location, is intended to include any one or more of: proximate, on, near, adjacent to or within the specific location. As used herein, the terms proximal is defined with respect to an object, element, or user. For example, the term proximal may refer to the part or portion closer to the user.

The term nonwoven as used in the specification may refer to a material made from continuous (long) filaments (fibers) and/or discontinuous (short) filaments (fibers) by processes such as spunbonding, meltblowing, wetlaying, carding, airlaid and the like. Nonwovens do not have a woven or knitted filament pattern. It is to be appreciated that nonwovens having various basis weights can be used in accordance with the methods herein. For example, some nonwovens may have basis weight of about 7 gsm to about 100 gsm, or more preferably between 15 gsm and 90 gsm. In another aspect nonwovens may have a basis weigh of about 7 gsm to about 120 gsm. All of the aforementioned examples and may include all 1 gsm increments within the above-recited range and all ranges formed therein.

Substrate as used in the specification may describe a material which is primarily two-dimensional (i.e. in an XY plane) and whose thickness (in a Z direction) is relatively small (i.e. 1/10 or less) in comparison to its length (in an X direction) and width (in a Y direction). Non-limiting examples of substrates include a web, layer or layers or fibrous materials, nonwovens, films and foils. These materials may be used alone or may comprise two or more layers laminated or otherwise joined together. As such, a web is a substrate.

### II. Detailed Examples

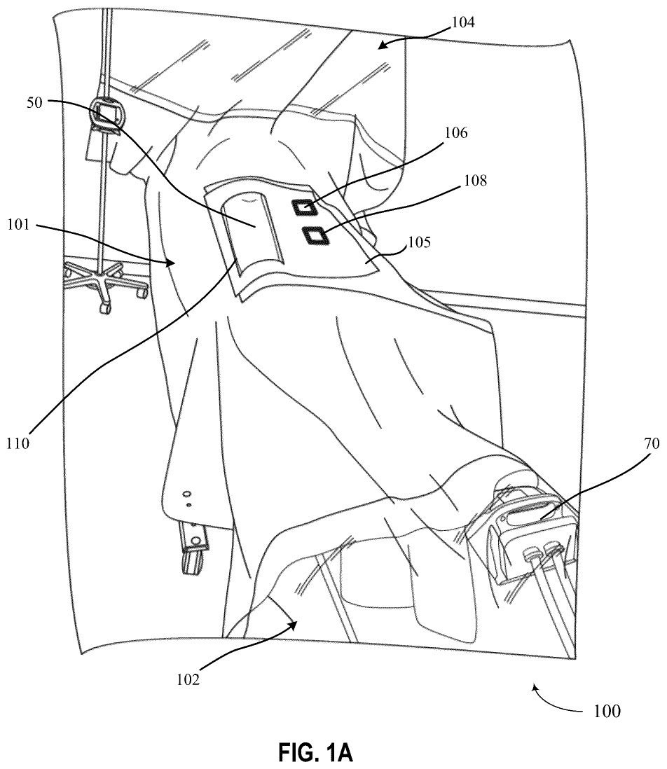

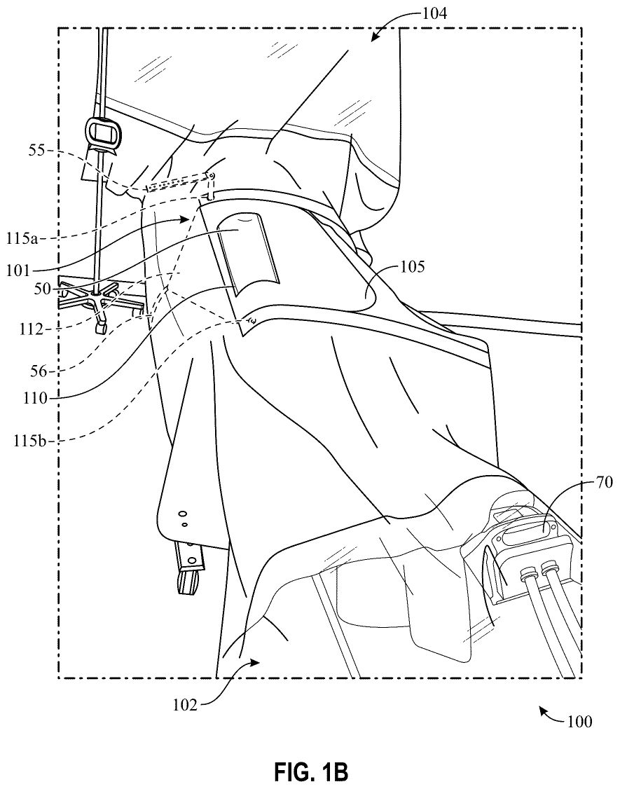

FIGS. 1A-3 show example of a drape or drape system according to aspects of the disclosure in an example in-use configuration. The drape 100 may be used to cover a patient with a leg 50 and an operating table and/or operating equipment 70. In some examples, the operating table may be a fracture table, a Hana® orthopedic surgery table, or other surgical table (e.g., a flat surgical table). The drape 100 may include a fenestration or opening 110 for providing access to a surgical site, such as a patient's leg 50. The drape may include a main body portion 101. The main body portion may include or have connected thereto a reinforcement portion 105 surrounding the opening 110. The drape 100 may further include one or more pockets or other instrument holders 106 and/or 108, which may be a separate component that can be adhered to the drape 100 prior to or during a procedure. The drape 100 may further include a first anesthesia screen 104 and/or a second anesthesia screen 102. The first anesthesia screen 104 and/or the second anesthesia screen 102 may be formed of a translucent material or non-opaque or semi-opaque material to allow a technician and/or for a practitioner to view equipment (i.e., operating equipment 70) through the first anesthesia screen 104 and/or second anesthesia screen 102.

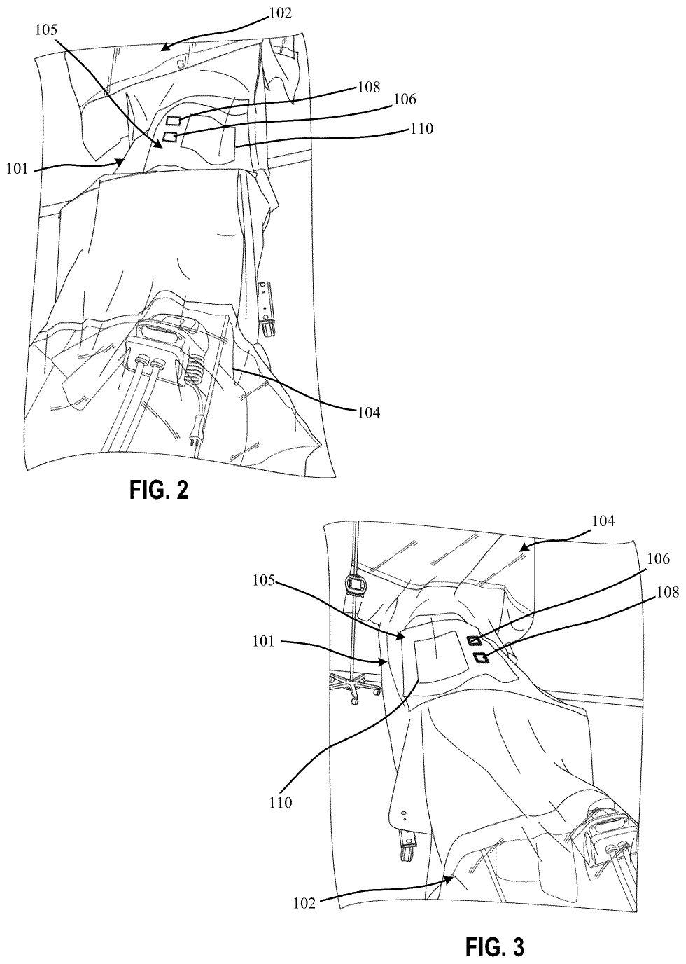

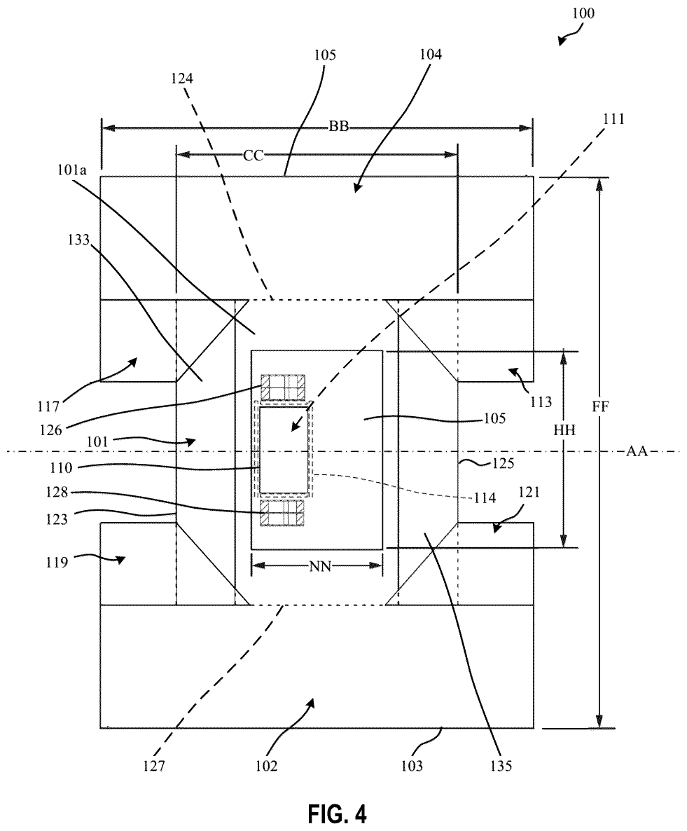

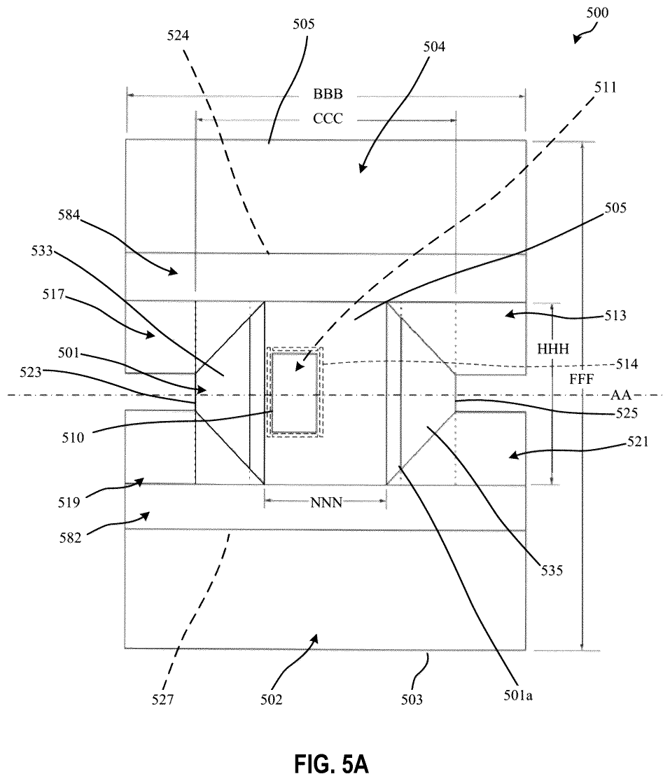

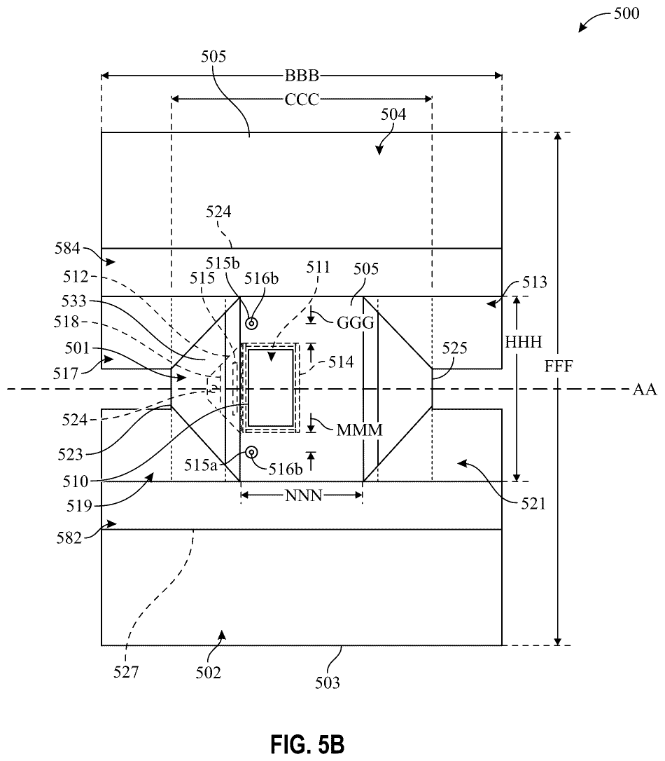

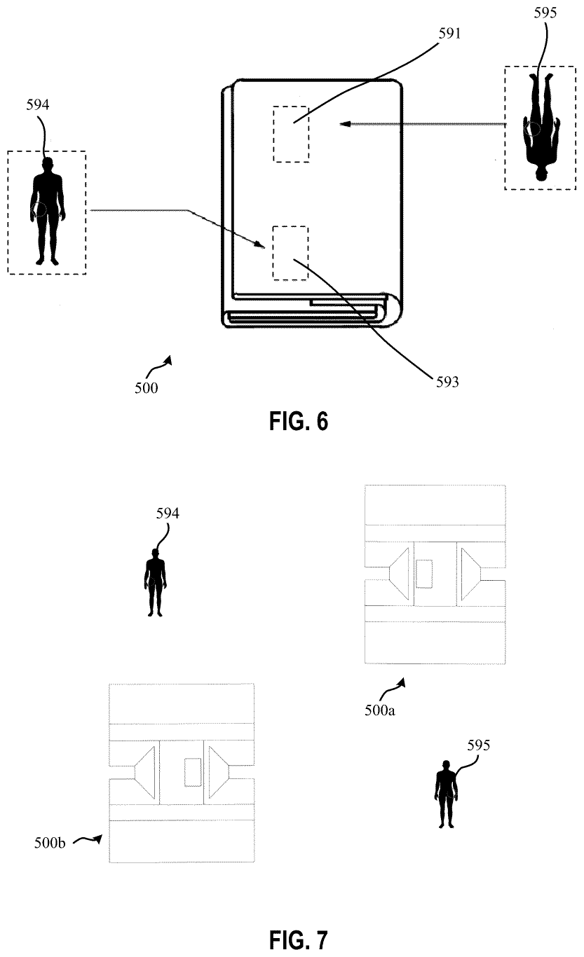

As shown in FIGS. 2 and 3, the drape 100 may be symmetrical or substantially symmetrical about an axis (e.g., axis AA in FIGS. 4 and 5), enabling the drape 100 to be used on a procedure on a right leg of a patient (e.g., as shown in FIG. 2) or a left leg of a patient (e.g., as shown in FIG. 3). In other words, the drape may be an ambipedal (i.e., capable of being used for surgical procedures on either leg of a patient).

FIG. 4 shows an example of a drape 100 according to aspects of the disclosure. The drape 100 may share any one or any combination of features with the drapes described herein. The drape 100 may for example include a main body portion 101. The main body portion 101 may be formed of one or more layers of material. Some example materials include but are not limited to a substrate of spunbond and meltblown fibers forming “layers” of the material. For example, the main body portion 101 may be formed of a first and second layer of spunbond fibers with a layer of meltblown fibers therebetween (which may hereinafter be referred to as an “SMS” material). Each of the spunbond and/or meltblown layers may be formed from a series of fibers, bonded together by chemical, mechanical, heat, and/or solvent treatment. Further, the fibers of each of the spunbond and/or meltblown layers may be formed of any one of or a combination of polypropylene, polyethylene, polyester, polyimides, or any thermoplastic polymer, including biodegradable polymers, to name a few examples.

In one example implementation of the disclosure, the aforementioned spunbond layers may be composed of longer interlocked continuous filaments than the filaments forming the meltblown layer. The process of forming the spunbond layer may comprise steps of: filament extrusion; filament orientation; filament laydown; and web bonding. The spunbond process may for filaments from chips of any one of or combination of the aforementioned materials as the “material to be extruded.” The material to be extruded may then be provided to an extruder. Once the pellets are extruded a spin pump or spin pumps may form spinnerets from the extruded pellets. Cooling or operating air may then be provided to the spinnerets to at least partially solidify the extruded pellet material. The at least partially solidified spinnerets may then be supplied to a draw-off or laydown system, which provides the solidified fibers to a web forming belt. The web formed on the belt may then be provided to a bonder that bonds the filaments together. Methods of binding the individual fibers may include any one or a combination of applying binders; applying solvents; applying heat and/or pressure to melt or partially melt the individual fibers to one another and or providing a heated needle loom met individual fibers to one another. It is noted that while various processes and process variations have been given here as an example. The disclosed example above is not intended to be limiting and one or more of the layers of the main body portion main body portion 101 may be formed using the aforementioned processes and may be formed using any known method in the art.

The aforementioned meltblown layer or layer(s) may be produced by providing short, melted polymer fibers through a spin net and/or by providing the material through passages or multiple passages. The passages or die may be arranged so that the material, which may generally have a lower viscosity than the material provided during the spundbond layer is provided through the die and intersects with passages that provide heated and/or pressurized air and/or gas to the extruded fibers. The forces imparted on the material cause short fibers to be formed that are self-adhering and are provided to a collection screen, rotating collector, and/or meltblown web. The individual fibers may be fully or partially solidified on the collection screen, winder and/or web. Some other methods of forming the liquid barrier layer may include electrospinning, for example. The fibers of the meltblown layer or meltblown layer(s) may be significantly smaller in cross-section than the spundbond fibers. For example, the meltblown fibers of the meltblown layer may range between 0.05 and 10 micrometers. In another example, the individual fibers 109 of the meltblown layer may range between 0.05 and 10 micrometers with an average fiber diameter of 1 to 2 micrometers.

In yet another example, the individual fibers of the meltblown layer may range between 0.05 and 5 micrometers with an average fiber diameter of 1 to 2 micrometers. The small diameter of individual fibers of the meltblown layer may provide a barrier to prevent water or other liquids from passing through the meltblown layer. However, since the meltblown fibers have a very small average fiber diameter they may have low intrinsic strength. Thus, the aforementioned spunbond layer(s) may be added to the meltblown layer to provide strength to the substrate. Further, the meltblown layer may comprise a number of meltblown “layers” formed based on the number of beams of meltblown provided during the manufacturing process. It is noted that while the term “layers” is used a non-woven may actually not be layers with discrete boundaries therebetween but may be comprised of fibers that are intertwined between and connecting each “layer” during the manufacturing process. Further it is noted that while a SMS material is described here (i.e., two spundbond layers with a meltblown layer between the two spundbond layers), any number of “layers of either meltblown or spunbond is contemplated in this disclosure. For example, the non-woven material forming the main body portion 100 may include more than one meltblown “layer” and/or more than two spundbond layers, i.e., may be formed of a SMMS and/or SSMS material, or the like.

The material of the main body portion and/or the non-woven substrate described herein may further include a non-porous or microporous polypropylene layer such as a backsheet and/or as a layer laminated between layers of the nonwoven materials described above. The non-porous and/or microporous film may be laminated to the example nonwovens described above to strengthen the main body portion and/or to otherwise improve the sterile properties thereof.

It is noted that while nonwoven substrates are provided above as examples, the main body portion 101 may be formed of any known suitable material for a surgical environment and especially materials that are suitable for use in a surgical drape.

The main body portion 101 may have a fenestration or opening 110 therein for accessing a surgical site. The opening 110 may for example be a rectangular opening as shown in FIGS. 1A and 1B. In some examples, the opening may have first dimension (i.e., a width) of 5-20 inches. In another example the first dimension may be 7-14 inches. In yet another example, the first dimension may be approximately 10 inches. Further, in some examples the opening may have second dimension (i.e., a length) of 10-30 inches. In another example the second dimension may be 15-27 inches. In yet another example, the second dimension may be approximately 20 inches. It is noted that while the opening 110 is shown in FIG. 4 as a rectangular opening, any other opening or fenestration shape is contemplated in this disclosure. For example, the opening 110 may be circular, oval, oblong, trapezoidal, stadium-shaped or may be shaped as a rounded rectangle (i.e., rectangle that is chamfered or rounded at each corner).

The opening 110 may have at least one adhesive portion 114 proximal to or in proximity of the edge of the opening 110 on a bottom-side (i.e., a patient facing side) of the main body portion 101. The at least one adhesive portion 114 may include adhesive portions that surround or at least partially surround the opening 110. In one example, the at least one adhesive portion 114 may for example be comprised of a pressure sensitive adhesive or other adhesive configured to adhere to a patient's skin, while still allowing removal of the adhesive after a surgical procedure is complete. The adhesive portion(s) may for example include a release liner or other protective covering that is removable to expose the adhesive for adhering the border of the opening 110 to a patient in preparation for a procedure. Adhering drape 100 to the patient near or at the opening 110 may ensure a sterile field is maintained at the surgical site surrounded by the opening 110.

FIGS. 1B and 5B show another example of a drape according to aspects of this disclosure. Turning to FIG. 1B, the drape or drape system 100 shown in FIG. 1B may share features with or may be analogous with the drape 100 described above. For example, features described above that include like reference numbers may be similar to or identical to the features described above.

As noted above, the drape 100 may be used to cover a patient with a leg 50 and an operating table and/or operating equipment. The operating table may be a fracture table, a Hana® orthopedic surgery table, or other surgical table (e.g., a flat surgical table). In some examples, the table may include a lifting mechanism (hidden from view). The lifting mechanism may for example be a femur lift with a femoral hook support 55. As shown in the example implementation of FIG. 1B, the femur lift may for example have an extension portion that extends from the lifting mechanism along a femoral lift axis and that extends through the drape 100. The femoral hook support 55 may for example extend at an angle with respect to the extension portion. In some examples, the femoral hook support 55 may extend substantially orthogonally with respect to the femoral lift axis. The femoral lift support may for example include one or more support openings that are configured to have a femoral hook connected thereto. The femur lift support may further be rotatable about the femoral hook support axis.

In the example above, the drape 100 may include one or more accessory openings 115a and/or 115b that are configured to have accessories (e.g., the aforementioned femoral hook support extension portion) passed therethrough while maintaining a sterile field. As described in further detail below with respect to FIG. 5B, the one or more accessory openings 115a and/or one or more accessory openings 115b may for example have a seal or flexible inner portion that is heat sealed or otherwise sealingly connected (e.g., glued or otherwise adhered, solvent welded, friction welded and/or stitched or sewn) to surround openings in the drape 100. The seal or flexible inner portion may be formed of a flexible material with a central opening that is smaller than the accessory to be passed therethrough. The material may stretch or otherwise confirm to create a seal around the accessory that is passed through the opening. Examples of materials and/or features of the one or more accessory openings 115a and/or 115b are described in further detail below with respect to FIG. 5B. While not shown, the drape 100 may include a sock-like cover or other cover that is connected to the gown or configured to be connected to the gown. The sock-like cover may fully enclose or partially enclose the femoral hook support 55. The aforementioned sock-like cover may be implemented either in combination with the above-mentioned features of the one or more accessory openings 115a and/or 115b and/or as an alternative to any one or combination of the aforementioned features of the accessory openings 115a and/or 115b.

In some examples, the drape 100 or drape system 100 may include a fluid management system and/or utility pouch for managing fluid and/or other surgical needs. The fluid management system maybe aligned with or otherwise configured to collect fluid from a surgical site corresponding with the opening 110. In one example, the fluid management system includes a fluid management pouch 112. The fluid management pouch 112 may be pre-connected to the drape 100 via any known connection method (e.g., glued or otherwise adhered, solvent welded, friction welded and/or stitched or sewn). In another example, the fluid management pouch 112 may instead be included with the drape 100 as a separate component and may include an adhesive strip or other connection portion allowing a user to connect the fluid management pouch 112 to the drape 100 as desired. In some examples, the fluid management pouch 112 may include a pressure sensitive adhesive and a release liner or other protective covering that is removable to expose the adhesive for adhering pressure sensitive adhesive to the drape 100. Once the release liner is removed by a user, the user may adhere the fluid management pouch 112 to the drape 100 by pressing the pressure sensitive adhesive to the surface of the drape 100. In some examples, the drape 100 may include visual markings or indicators that may guide a user to adhere the fluid management pouch 112 to a specific location of the drape 100. For example, the drape 100 may include a printed rectangular box that corresponds with the edge of the opening 110. In some examples, the fluid management pouch may also function as a utility pouch for storing and/or managing surgical supplies and/or equipment. For example, the fluid management pouch may be used to manage large instruments and/or tubes during a procedure.

As shown in FIG. 1B, the fluid management pouch 112 may be triangular shaped or may otherwise have a wider portion that is proximal to the opening 110 and may narrow towards a location distal to the opening 110 to funnel fluids collected from the opening 110. The fluid management pouch 112 may have tubing 56 connected thereto and in fluid communication with the bottom portion of the fluid management pouch 112 to drain fluid from the fluid management pouch 112. Additional details of the fluid management pouch 112 are described in further detail below with respect to FIG. 5B.

The drape may further include a removable opening cover (not shown). The removable opening cover may for example be removably adhered to or otherwise removably connected proximal to or in proximity of the edge of the opening 110 on a top-side (i.e., the side of the drape 100 facing the technician or practitioner opposite the patient facing side) of the main body portion 101. The opening cover may for example be provided to cover the opening 110 prior to a surgical procedure (e.g., either before or after the at least one adhesive portion 114 is adhered to a patient). In some examples, the removable opening cover may be formed of an SMS material (e.g., formed of a material that shares features with or is identical to the material of the main body portion 101). The material of the opening cover may for example have a contrasting color to the surrounding portion of the main body portion 101 to clearly indicate to a user which portion of the gown is removable to expose the opening 110.

In some examples, the drape may include an incise drape or incise covering 111 that covers or otherwise expands across the opening 110. The incise drape 111 may for example be a transparent or semi-transparent flexible or semi-flexible member that is configured to adhere to a patient's skin at the surgical site within the opening 110. In some examples, the incise drape 111 may be connected to the bottom-side of the drape (i.e., the side that faces the patient) along a border of the opening 110. In another example, the incise drape 111 may be connected to the top-side of the drape (i.e., the side that faces away from the patient and/or that faces the practitioner) along a border of the opening 110.

The drape 100 may further include a reinforcement portion 105. In the example of FIG. 4, the reinforcement portion 105 is connected, laminated, and/or adhered to the main body portion 101. The reinforcement portion 105 may be formed of an SMS material that is analogous with or shares features with the examples of SMS materials described above with respect to the main body portion 101. In some examples, the reinforcement portion 105 may be formed of an identical material to the main body portion 101. The reinforcement portion 105 may for example be provided in an area surrounding the opening 110 and may provide reinforcement to the drape at or near the opening 110 to prevent tearing of the drape 100. The reinforcement portion 105 may further provide an area for having pockets or other instrument holders 126 and/or 108 mounted thereto, that may either be permanently mounted to the drape 100 and/or may be customizable or adhered to the drape 100 by a clinician prior to a procedure dependent on the clinician's preferences. The pockets or instrument holders 126 and/or 128 may for example have three sides adhered to the reinforcement portion 105 with an opening on an end to form a pouch or other pocket for removably retaining one or more instruments such as scalpels, scissors and/or suction instruments to name a few non-limiting examples. In some examples, the pockets 126 and/or 128 may be provided with an adhesive backing that may be configured to be removed by a clinician or technician and then adhered to the drape 100. The aforementioned example allows a clinician to place the pockets wherever practical or preferrable. The instrument holders 126 and/or 128 may cither in combination with the aforementioned features or as an alternative to the aforementioned features may include one or more loops or other retaining features for retaining instruments or other supplies that may be used during a surgical procedure. In some examples, the pockets or instrument retaining features 126 and/or 128 may be permanently adhered to the drape 100 and may come fixed to the drape.

In some non-limiting examples, the reinforcement portion 105 may have width dimension NN. The width dimension NN may be 25-55 inches. In another example, dimension NN may be 30-50 inches. In yet another example, dimension NNN may be approximately 40 inches. Further, in some examples the reinforcement portion 105 may have length dimension HH. The length dimension HH may be 40-75 inches. In another example, dimension HH may be 50-70 inches. In yet another example, dimension HH may be approximately 61 inches.

The drape 100 may further include a plurality of armboard covers. The armboard covers may be connected to the main body portion 101. The drape 100 may for example include a first armboard cover 113, a second armboard cover 117, a third armboard over 119, and a fourth armboard cover 121. In some examples, the first armboard cover 113 and the second armboard cover 117 may be connected to the main body portion 101 and a first anesthesia drape 104 (described in further detail below). In some examples, the connection between the first armboard cover 113 and the second armboard cover 117 and the main body portion 101 may be along a diagonal as shown in FIG. 4. Further, in some examples, the third armboard cover 113 and the fourth armboard cover 121 may be connected to a second anesthesia drape 104 (described in further detail below), and may further be connected to the main body portion 101. In some examples, the connection between the third armboard cover 119 and the fourth armboard cover 121 and the main body portion 101 may also be along a diagonal as shown in FIG. 5A or 5B.

Any one or combination of the first armboard cover 113, second armboard cover 117, third armboard cover 119, and/or fourth armboard cover 121 may be formed of a SMS material that may include features of or may be analogous with the SMS material(s) described above with respect to the main body portion 101. In some examples, any one or combination of the first armboard cover 113, second armboard cover 117, third armboard cover 119 and/or fourth armboard cover 121 may be formed as a unitary structure (i.e., as a single sheet) with the main body portion 101. In another example, any one or combination of the first armboard cover 113, second armboard cover 117, third armboard cover 119 and/or fourth armboard cover 121 may glued or otherwise adhered to (e.g., friction, chemical, and/or heat sealed or adhered) to a respective portion of the main body portion 101. The aforementioned armboard covers may provide the advantage of covering one or more of the armboards of a surgical bed or hospital bed to ensure a sterile field is maintained during a surgical procedure.

The drape 100 may further include one or more anesthesia screens 104 and/or 102. For example, a first anesthesia screen 104 may be connected to the main body portion 101 at or near a first edge 124 of the main body portion (which may be interchangeably referred to herein as a third side portion). The drape 100 may further include a second anesthesia screen 102 that may be connected to a second edge 127 of the main body portion 101 (which may be interchangeably referred to herein as a fourth side portion). Any one or both of the aforementioned anesthesia screens 104 and/or 102 may be formed of a clear, semi-clear, or semi-opaque material. For example, the anesthesia screens 104 and/or 102 may be formed of a clear or semi-clear plastic such as a polyolefin (e.g., polyethylene) or the like. In some examples the one or more anesthesia screens 104 and/or 102 may be connected to the main body portion 101 via any one or combination of an adhesive or other bonding method such as heat sealing, friction welding or the like. The aforementioned anesthesia screens may allow for viewing and/or access to operating equipment 70 (FIG. 1A or 1B) or other areas that are desired to be viewable during a surgical procedure while still allowing a sterile field to be maintained. In one example each of the anesthesia screen may include a separate sheet of opaque material or non-transparent material connected at an edge of the anesthesia screen and/or the edge of the main body portion 101 (e.g., at or near the first edge 124 and/or second edge 127), which may allow a technician or clinician to instead utilize a non-transparent drape either as an alternative to or in conjunction with the aforementioned anesthesia drapes 104 and/or 102 based on preference.

While dimensions are not intended to be limiting, FIG. 4 shows examples of dimensions that may be applicable to aspects of the disclosure. The width 4BB of the drape 500. In some examples, dimension 4BB may be 80-200 inches. In another example, dimension 4BB may be 90-150 inches. In yet another example, dimension 4BB may be approximately 124 inches.

Further, a width of a center portion of the drape 100 may have a dimension CC. The dimension CC may for example be 100-150 inches, in another example, dimension CC may be 90-100 inches. In yet another example, dimension CC may be approximately 86 inches.

FIG. 4 further shows an example width FF of the drape 100. In some examples, dimension FF may be 90-250 inches. In another example, dimension FF may be 120-180 inches. In yet another example, dimension FF may be 159-167 inches.

FIGS. 5A and 5B shows another examples of a drape 500 according to aspects of the disclosure. The drape 500 may share any one or any combination of features with the drape 100 of FIG. 4 and/or the drape 100 of FIGS. 1A-3. The drape 500 may for example include a main body portion 501. The main body portion 501 may be formed of one or more layers of material. Some example materials include but are not limited to a substrate of spunbond and meltblown fibers forming “layers” of the material. For example, the main body portion 501 may be formed of a first and second layer of spunbond fibers with a layer of meltblown fibers therebetween (which may hereinafter be referred to as an “SMS” material). The SMS or other material of the drape 500 and/or main body portion 501 may include aspects of or may be analogous with the materials described above with respect to FIG. 4.

It is noted that while nonwoven substrates are provided above as examples, the main body portion 501 may be formed of any known suitable material for a surgical environment especially materials that are suitable for use in a surgical drape.

The main body portion 501 may have a fenestration or opening 510 therein for accessing a surgical site. The opening 510 may for example be a rectangular opening as shown in FIGS. 5A and 5B. In some examples the opening may have first dimension (i.e., a width) of 5-20 inches. In another example the first dimension may be 7-14 inches. In yet another example, the first dimension may be approximately 10 inches. Further, in some examples the opening may have second dimension (i.e., a length) of 10-30 inches. In another example the second dimension may be 15-27 inches. In yet another example, the second dimension may be approximately 20 inches. It is noted that while the opening 510 is shown in FIGS. 5A and 5B as a rectangular opening, any other opening or fenestration shape is contemplated in this disclosure. For example, the opening 510 may be circular, oval, oblong, trapezoidal, stadium-shaped or may be shaped as a rounded rectangle (i.e., rectangle that is chamfered or rounded at each corner).

The opening 510 may have at least one adhesive portion 514 proximal to or in proximity of the edge of the opening 510 on a bottom-side (i.e., a patient facing side) of the main body portion 501. The at least one adhesive portion 514 may include adhesive portions that surround or at least partially surround the opening 510. In one example the at least one adhesive portion 514 may for example be comprised of a pressure sensitive adhesive or other adhesive configured to adhere to a patient's skin, while still allowing removal of the adhesive after a surgical procedure is complete. The adhesive portion(s) may for example include a release liner or other protective covering that is removable to expose the adhesive for adhering the border of the opening 510 to a patient in preparation for a procedure. Adhering drape 500 to the patient near or at the opening 510 may ensure a sterile field is maintained at the surgical site surrounded by the opening 510.

The drape may further include a removable opening cover (not shown). The removable opening cover may for example be adhered to or otherwise removably connected proximal to or in proximity of the edge of the opening 510 on a top-side (i.e., the side of the drape 500 facing the technician or practitioner opposite the patient facing side) of the main body portion 501. The opening cover may for example be provided to cover the opening 510 prior to a surgical procedure (e.g., either before or after the at least one adhesive portion 514 is adhered to a patient). In some examples, the removable opening cover may be formed of an SMS material (e.g., formed of a material that shares features with or is identical to the material of the main body portion 501). The material of the opening cover may for example have a contrasting color to the surrounding portion of the main body portion 501 to clearly indicate to a user which portion of the gown is removable to expose the opening 510.

In some examples, the drape may include an incise drape or incise covering 511 that covers or otherwise expands across the opening 510. The incise drape 511 may for example be a transparent or semi-transparent flexible or semi-flexible member that is configured to adhere to a patient's skin at the surgical site within the opening 110. In some examples, the incise drape 511 may be connected to the bottom-side of the drape (i.e., the side that faces the patient) along a border of the opening 510. In another example, the incise drape 511 may be connected to the top-side of the drape (i.e., the side that faces away from the patient and/or that faces the practitioner) along a border of the opening 510.

The drape 500 may further include a reinforcement portion 505. In the example of FIGS. 5A and 5B, the reinforcement portion 505 is connected, laminated, and/or adhered to the main body portion 501. The reinforcement portion 505 may be formed of an SMS material that is analogous with or shares features with the examples of SMS materials described above with respect to the main body portion 501. In some examples, the reinforcement portion 505 may be formed of an identical material to the main body portion 501. The reinforcement portion 505 may for example be provided in an area surrounding the opening 510 and may provide reinforcement to the drape at or near the opening 510 to prevent tearing of the drape 500. The reinforcement portion 505 may further provide an area for having pockets or other instrument holders mounted thereto (not show in FIG. 5A or 5B, examples shown in FIG. 4 as references 126 and 128) that may either be permanently mounted to the drape 500 and/or may be customizable or adhered to the drape 500 by a clinician prior to a procedure dependent on their preferences. In some non-limiting examples, the reinforcement portion 505 may have width dimension NNN. The width dimension NNN may be 25-55 inches. In another example, dimension NNN may be 30-50 inches. In yet another example, dimension NNN may be approximately 40 inches. Further, in some examples the reinforcement portion 505 may have length dimension HHH. The length dimension HHH may be 40-75 inches. In another example, dimension HHH may be 50-70 inches. In yet another example, dimension HHH may be approximately 61 inches.

The example implementations shown in FIGS. 5A and 5B further include a first outer main body portion 584 and a second outer main body portion 582. The first outer main body portion 584 may be connected to a first side of the main body portion 501 and the second outer main body portion 582 may be connected to the second side of the main body portion 501. The first outer main body portion 584 and/or the second outer main body portion 582 may be formed of a SMS material that may include features of or may be analogous with the SMS material(s) described above with respect to the main body portion 501. In some examples, the first outer main body portion 584 and/or second outer main body portion 582 may be formed as a unitary structure (i.e., as a single sheet) with the main body portion 501. In another example, the first outer main body portion 584 and/or second outer main body portion 582 may glued or otherwise adhered to (e.g., friction, chemical, and/or heat sealed or adhered) to the first and second sides of the main body portion 501 as shown in FIGS. 5A and 5B.

The drape 500 may further include a plurality of armboard covers. The armboard covers may be connected to the main body portion 501 and/or the first outer main body portion 584 and the second outer main body portion 582. The drape 500 may for example include a first armboard cover 513, a second armboard cover 517, a third armboard over 519, and a fourth armboard cover 521. In some examples, the first armboard cover 513 and the second armboard cover 517 may be connected to the first outer main body portion 584 and may further be connected to the main body portion 501. In some examples, the connection between the first armboard cover 513 and the second armboard cover 517 and the main body portion 501 may be along a diagonal as shown in FIGS. 5A and 5B. Further, in some examples, the third armboard cover 513 and the fourth armboard cover 521 may be connected to the second outer main body portion 582 and may further be connected to the main body portion 501. In some examples, the connection between the third armboard cover 519 and the fourth armboard cover 521 and the main body portion 501 may also be along a diagonal as shown in FIGS. 5A and 5B.

Any one or combination of the first armboard cover 513, second armboard cover 517, third armboard cover 519, and/or fourth armboard cover 521 may be formed of a SMS material that may include features of or may be analogous with the SMS material(s) described above with respect to the main body portion 501. In some examples, any one or combination of the first armboard cover 513, second armboard cover 517, third armboard cover 519 and/or fourth armboard cover 521 may be formed as a unitary structure (i.e., as a single sheet) with the main body portion 501 and/or the respective one of the first outer main body portion 584 and/or second outer main body portion 582. In another example, any one or combination of the first armboard cover 513, second armboard cover 517, third armboard cover 519 and/or fourth armboard cover 521 may glued or otherwise adhered to (e.g., friction, chemical, and/or heat sealed or adhered) to a respective portion of the main body portion 501 and/or the first outer main body portion 584 and/or second outer main body portion 582. The aforementioned armboard covers may provide the advantage of covering one or more of the armboards of a surgical bed or hospital bed to ensure a sterile field is maintained during a surgical procedure.

The drape 500 may further include one or more anesthesia screens 504 and/or 502. For example, a first anesthesia screen 504 may be connected to the first outer main body portion 584 at or near a first edge of the first outer main body portion 584 (which may be interchangeably referred to herein as a third side portion). The drape 500 may further include a second anesthesia screen 502 that may be connected to the second outer main body portion 582 at or near a first edge of the second outer main body portion 526 (which may be interchangeably referred to herein as a fourth side portion). Any one or both of the aforementioned anesthesia screens 504 and/or 502 may be formed of a clear, semi-clear, or semi-opaque material. For example, the anesthesia screens 504 and/or 502 may be formed of a clear or semi-clear plastic such as a polyolefin (e.g., polyethylene) or the like. In some examples the one or more anesthesia screens 504 and/or 502 may be connected to the first outer main body portion 584 and/or the second outer main body portion 582 via any one or combination of an adhesive or other bonding method such as heat sealing, friction welding or the like. The aforementioned anesthesia screens may allow for viewing and/or access to operating equipment 70 (FIG. 1) or other areas that are desired to be viewable during a surgical procedure while still allowing a sterile field to be maintained.

While dimensions are not intended to be limiting, FIGS. 5A and 5B show examples of dimensions that may be applicable to aspects of the disclosure. The drape 500 may have a width BBB. In some examples, BBB may be 80-200 inches. In another example, dimension BBB may be 90-150 inches. In yet another example, dimension BBB may be approximately 124 inches.

Further, a width of a center portion of the drape (i.e., the portion that spans from the main body first side portion 523 to the main body second side portion 525) may have a dimension CCC. The dimension CCC may for example be 100-150 inches, in another example, dimension CCC may be 90-100 inches. In yet another example, dimension CCC may be approximately 86 inches.

FIGS. 5A and 5B further show example widths FFF of the drape 500. In some examples, dimension FFF may be 90-250 inches. In another example, dimension FFF may be 120-180 inches. In yet another example, dimension FF may be 159-167 inches.

FIG. 5B illustrates a drape system with additional features that may be implemented with any of the features described herein. The drape or drape system 500 shown in FIG. 5B may share features with or may be analogous with the drape 100 and/or the drape or drape system 500 described above. For example, features that include like reference numbers may be similar to or identical to the features described above.

As discussed above with respect to FIG. 1B, the drape 500 may further include one or more accessory openings 515a and/or 515b that are configured to have accessories (e.g., the aforementioned femoral hook support extension portion) passed therethrough while maintaining a sterile field. The one or more accessory openings 515a and/or one or more accessory openings 515b may for example have a seal or flexible inner portion 516a and/or 516b that is heat sealed or otherwise sealingly connected (e.g., glued or otherwise adhered, solvent welded, friction welded and/or stitched or sewn) at or near openings in the drape 500. In some examples, the one or more accessory openings 515a and/or 515b may be openings in the reinforcement portion 505 of the drape 500. The seal or flexible inner portion may be formed of a flexible material with a central opening that is smaller than the accessory to be passed therethrough (e.g., the femoral hook support 55 and/or the femoral hook support extension portion shown in FIG. 1B). The material of the seal or flexible inner portion 516a and/or 516b may be configured to stretch or otherwise confirm to create a seal around the accessory that is passed through the opening. In some examples, the one or more accessory openings 515a and/or 515b may be formed of an elastomeric material that exhibits rubber-like elasticity and can stretch and return to its original state such as an elastomeric film. While not intended to be limiting, an elastomer may include any known material with viscoelasticity and with weak intermolecular forces (i.e., generally low Young's modules) and high failure strain. Some examples of materials that can be used to form the flexible inner portions 516a and/or 516b include but are not limited to any one or combinations of ethylene propylene rubber (EPM and EPDM), epichlorohydrin rubber, epichlorohydrin rubber, silicone rubber, fluorosilicone rubber, fluoroelastomers, perfluoroelastomers, polyether block amides, chlorosulfonated polyethylene, ethylene-vinyl acetate, thermoplastic elastomers, Poly(dichlorophosphazene), polyisoprene, polybutadiene, chloroprene, butyl rubber, styrene-butadiene rubber, nitrile rubbers and the like.

In some examples, the seal or flexible inner portion 516a and/or 516b may be formed of a Kraton®, a styrenic block copolymer (SBC) produced by Kraton Corporation. While not shown, the drape 500 may include a sock-like cover or other cover that is connected to the gown or configured to be connected to the gown. The sock-like cover may fully enclose or partially enclose the femoral hook support 55. The aforementioned sock-like cover may be implemented either in combination with the above-mentioned features of the one or more accessory openings 115a and/or 115b and/or as an alternative to any one or combination of the aforementioned features of the accessory openings 115a and/or 115b.

The drape may further include a removable accessory opening cover or covers that cover (not shown) that convert either one of or both of the one or more accessory openings 515a and/or 515b. The removable opening cover(s) may for example be removably adhered to or otherwise removably connected proximal to or in proximity of the edge of the one or more accessory openings 515a and/or 515b on a top-side (i.e., the side of the drape 500 facing the technician or practitioner opposite the patient facing side) of the main body portion 501. The opening cover may for example be provided to cover the one or more accessory openings 515a and/or 515b prior to a surgical procedure and/or may be left in place in situations where either one of or both of the one or more accessory openings 515a and/or 515b is not used. In some examples, the removable opening cover may be formed of an SMS material (e.g., formed of a material that shares features with or is identical to the material of the main body portion 501). The material of the opening cover may for example have a contrasting color to the surrounding portion of the main body portion 501 and/or reinforcement portion 505 to clearly indicate to a user which portion of the gown is removable to expose the one or more accessory openings 515a and/or 515b.

In some non-limiting examples, the center of the one or more accessory openings 515a and/or 515b may be located distances MMM and GGG, respectively from edges of the opening 510. Either one of or both of the dimension(s) MMM and/or GGG may be 5-15 inches. In another example, either one of or both of the dimension(s) MMM and/or GGG may be 7-13 inches. In yet another example, dimension MMM and/or GGG may be approximately 10 inches.

In some examples, the drape 500 or drape system 500 may include a fluid management system. The fluid management system may be aligned with or otherwise configured to collect fluid from a surgical site corresponding with the opening 510. In one example, the fluid management system includes a fluid management pouch 512. The fluid management pouch 512 may be pre-connected to the drape 500 via any known connection method (e.g., glued or otherwise adhered, solvent welded, friction welded and/or stitched or sewn). In another example, the fluid management pouch 512 may instead be included with the drape 500 as a separate component and may include an adhesive strip or other connection portion allowing a user to connect the fluid management pouch 512 to the drape 500 as desired. In some examples, the fluid management pouch 112 may include a pressure sensitive adhesive and a release liner or other protective covering that is removable to expose the adhesive for adhering pressure sensitive adhesive to the drape 500. Once the release liner is removed by a user, the user may adhere the fluid management pouch 512 to the drape 500 by pressing the pressure sensitive adhesive to the surface of the drape 500. In some examples, the drape 500 may include visual markings or indicators that may guide a user to adhere the fluid management pouch 512 to a specific location of the drape 500. For example, the drape 500 may include a printed rectangular box that corresponds with the edge of the opening 510.

As shown in FIG. 1B, the fluid management pouch 112 may be triangular shaped or may otherwise have a wider portion that is proximal to the opening 510 and may narrow towards a location distal to the opening 510 to funnel fluids collected from the opening 510. The fluid management pouch 512 may have an opening 524 at or near the bottom thereof and may either be configured to have tubing (e.g., tubing 56 shown in FIG. 1B) connected to the opening 524 or may have tubing pre-connected or permanently connected to the opening 524 (tubing not shown in FIG. 5B, but an example of tubing 56 is shown in FIG. 1B). The opening 524 and tubing connected to the opening may be configured to drain fluid from the fluid management pouch 512.

The fluid management pouch 512 may further include a malleable strip 515 that allows a user to create a propped opening between the front surface and back surface of the fluid management pouch. The malleable strip 515 may for example be an aluminum or metallic strip that is incorporated into the material of the pouch 512. In some examples, the malleable strip 515 may for example be formed of an elastic material that is configured to prop-open the fluid management pouch 512 to ensure any fluid from the surgical site naturally drains into the fluid management pouch 512.

In some examples, the fluid management pouch 512 may further include a filter element 518. The filter element may be a mesh or material with openings therein to prevent any larger items that may fall into or otherwise drain into the fluid management pouch 512 from clogging the opening 524 of the fluid management pouch 512.

As mentioned above, both the drape 100 described above with respect to FIGS. 1-4 and drape 500 described above with respect to FIGS. 5A and/or 5B may be symmetrical or substantially symmetrical about an axis AA (FIGS. 4 and 5), thus allowing the drape to be used both on surgeries on a right side of the patient and on a left side of the patient. The symmetrical or substantially asymmetrical drape design improves versatility of the drape and eliminates the need for multiple types of drapes for surgeries of the right or left legs or hips, for example.

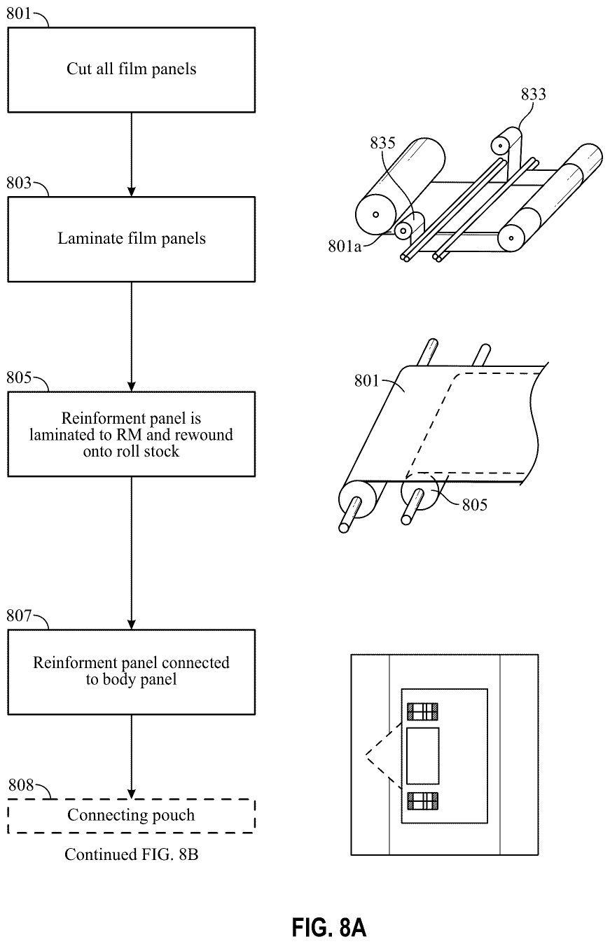

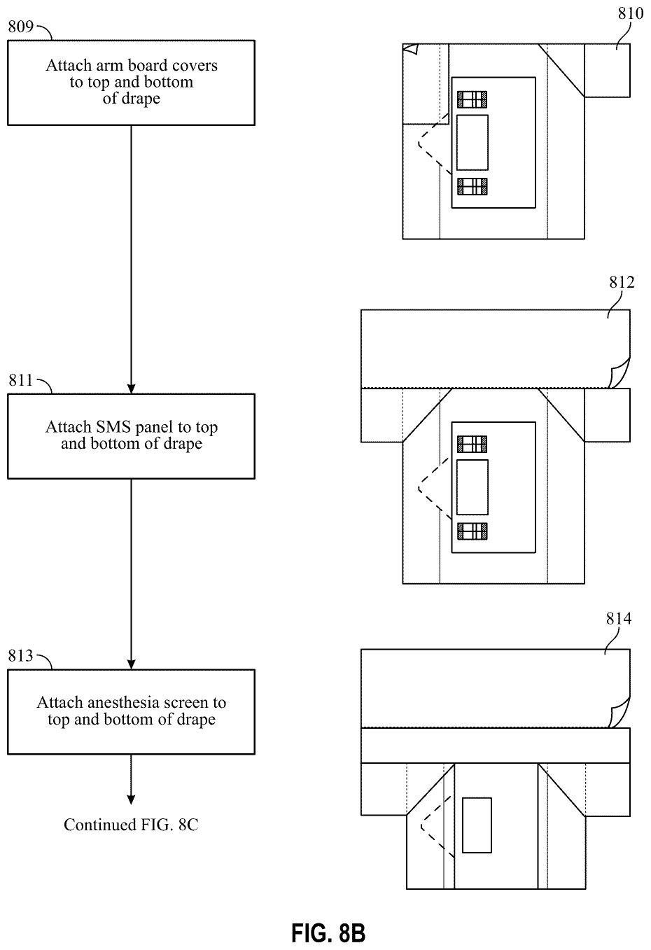

FIGS. 6 and 7 show one example of a folded drape that may share features with and/or may be analogous with drape 100 and/or 500 described above. Folded drape 500 may have indicators 591 and/or 593 to indicate a correct orientation of the drape for unfolding prior to a procedure. For example, a first indicator 591, may show a right hip 595 of a patient to show that if the drape is oriented in a first orientation (e.g., with the patient in the same orientation as the indicator, the drape 500 can be unfolded in a first orientation 500b (e.g., with the opening or fenestration at a right hip or leg of the patient. Further, a second indicator 593 may indicate a left hip 594 and thus when the drape 500 is unfolded with the drape in a second orientation the drape 500 can be unfolded in a second orientation 500a (e.g., with the opening of the fenestration at a left hip or leg of the patient). The indicators 591 and/or 593 may help to ensure that a sterile field is maintained (e.g., the top surface of the drape that faces away from the patient remains sterile during the unfolding process).

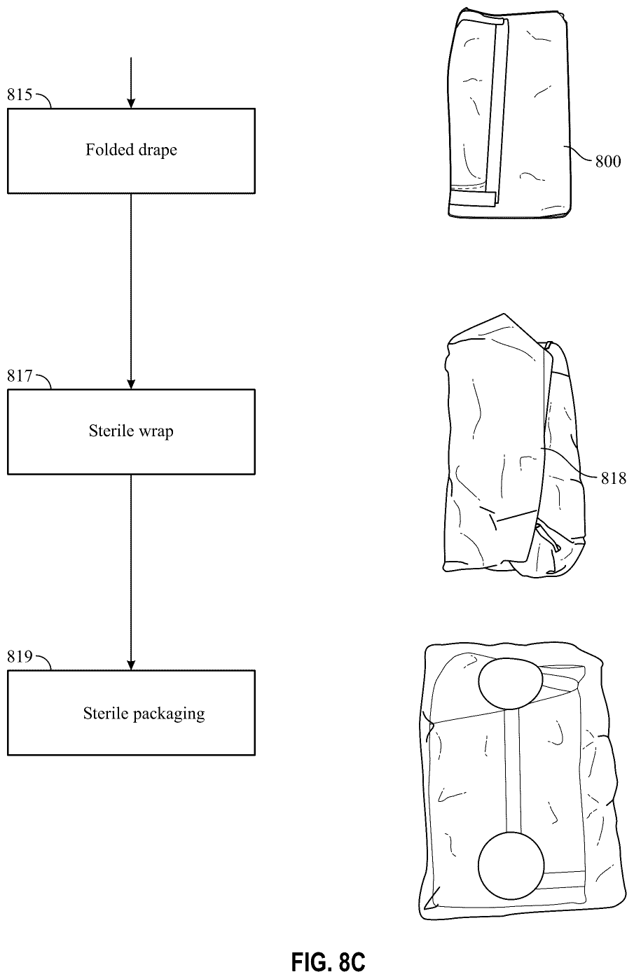

FIGS. 8A-8C are flowcharts and example images showing non-limiting examples of a manufacturing and/or assembly process of a drape (e.g., drape 100 and/or 500 described above) with respect to aspects of the disclosure. It is noted that the steps described do not exclude additional steps that may be added at any time during the assembly process. Further, steps described herein may be omitted without departing from the scope of the disclosure.

As shown in FIG. 8A, a step of manufacturing a drape may include cutting or providing panels of materials for producing the drape 100 and/or 500 described above. It is noted that example drape 800 described below may share features with may be analogous with either one of or both of drapes 100 and/or 500 described above. Further, the panels of material may include features of or may be analogous with the materials described above. In the example shown in FIG. 8A, the aforementioned materials may be provided in rolls. For example, the film panels (e.g., SMS materials described above) may be provided as a first roll. The first roll may include a first stock material 833 that may eventually form a first panel of the main body portion 801. The aforementioned first panel may share features with or may be analogous with the first side panel 133 shown in FIGS. 1A and 1B and/or the first side panel 533 shown in FIGS. 5A and/or 5B. Further, the aforementioned main body portion 801 may for example share features with or may be analogous with either one of or both the main body portions 101 and/or 501 described above, with a center panel formed of a center panel material 801a. The center panel may be analogous with or share features with either one of or both the center panels 101a and/or 501a shown in FIGS. 4 and 5.

A third roll may include a third stock material 835 that may eventually form a third panel or second side panel of the main body portion 801. The first panel may share features with or may be analogous with the second side panel 135 shown in FIGS. 1A and 1B and/or the second side panel 535 shown in FIGS. 5A and/or 5B. The first stock material 833, center panel material 801a, and second side panel material 835 are all laminated or otherwise joined together to form the main body portion (e.g., 101 and/of 501) of the drape in step 803. The panels may be connected via a glue, hot-melting, and/or friction welding.

In step 805, a reinforcement panel 805 is joined or laminated to the main body portion 801. As noted above, the main body portion 801 may share features with or may be analogous with the main body portion 101 and/or 501 described above. Further, the reinforcement panel 805 may share features with or may be analogous with the reinforcement portion 105 and/or reinforcement portion 505 described above.

Further, during any of the aforementioned steps, the seal or flexible inner portion(s) 516a and/or 516b may be laminated or otherwise connected to the main body and/or the reinforcement panel 805.

Step 807 shows the reinforcement panel connected to the main body portion. The opening or fenestration (e.g., opening 110 and/or opening 510) and/or one or more accessory openings 515a and/or 515b may be cut or otherwise removed from both the main body portion 801 and/or the reinforcement panel 805.

In optional step 808, the pouch (e.g., fluid management pouch 112 and/or 512 described above may be connected to or otherwise fastened to the reinforcement panel 805 and/or the main body portion 801.

In step 809, the armboard cover(s) 810 may be connected (e.g., using any of the connection methods described herein) to at each side of the main body portion 801. The armboard cover(s) 810 may share features with or may be analogous with the armboard cover(s) 117, 113, 119, and/or 121 of FIG. 4 and/or the armboard cover(s) 517, 513, 519, and/or 521 of FIGS. 5A and/or 5B.

In step 811, an outer panel 812 may be connected to either one or both sides of the drape. The outer panel 812 may for example share features with or may be analogous with any one or both first outer main body portion 584 and/or the second outer main body portion 582 of FIGS. 5A and/or 5B.

In step 813, the anesthesia screen 814 and/or the opaque anesthesia screen may be connected to one or both sides of the gown. The anesthesia screen 814 may share features with or may be analogous with anesthesia screens 104 and/or 102 of FIG. 4 and/or anesthesia screens 504 and/or 502 of FIGS. 5A and/or 5B.

In step 815, after the drape 800 is assembled, it may be folded. In some examples, the folded drape may include the indicators 591 and/or 593 as described above with respect to FIG. 6. Further, in the example implementation where the fluid control pouch is a separate component that is configured to be adhered to the drape 800 by the user, the fluid control pouch (e.g., fluid management pouch 112, and/or 512 may be placed onto or folded into the drape.

In step 817, once the drape 800 is folded, it may be wrapped in a sterile wrap 818. The sterile wrap may include any of the materials described herein.

In Step 819, the drape 800 and sterile wrap 818 may be packaged in sterile packaging, the sterile packaging may include any one or combination of the materials described herein. The sterile packaging may be sterilized and/or may be subject to a high-level disinfection step.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described herein that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

What is claimed is:

1. A surgical drape comprising:
a main body portion with a fenestration for accessing a surgical site, the main body portion comprising:
a main body first side portion;
a main body second side portion opposite the first side portion;
a main body third side portion extending in a direction substantially perpendicular to the main body first side portion and the main body second side portion;
a main body fourth side portion opposite the main body third side portion and extending in a direction substantially perpendicular to the main body first side portion and the main body second side portion; the surgical drape further comprising:
a first anesthesia screen extending away from the third side portion; and
a second anesthesia screen extending away from the fourth side portion.

2. The surgical drape of claim 1, wherein the fenestration is positioned on the main body portion closer to the first side portion than the second side portion.

3. The surgical drape of claim 1, further comprising a reinforcement portion at least partially surrounding the fenestration.

4. The surgical drape of claim 1, further comprising at least one pocket for retaining one or more surgical instruments.

5. The surgical drape of claim 4, further comprising a second pocket for retaining one or more surgical instruments.

6. The surgical drape of claim 4, wherein the pocket is adhesively adhered to the main body portion.

7. The surgical drape of claim 4, further comprising one or more pockets that have an adhesive backing and are configured to be adhered to the drape.

8. The surgical drape of claim 1, wherein the main body portion, the first anesthesia screen, and the second anesthesia screen are symmetrical about an axis that bisects the main body portion.

9. The surgical drape of claim 1, wherein the main body portion is formed of a non-woven material and the first anesthesia screen and the second anesthesia screen are formed of a transparent material.

10. The surgical drape of claim 1, further comprising a first armboard cover portion and a second armboard cover portion connected to the first anesthesia screen and a third armboard cover portion and fourth armboard cover portion connected to the second anesthesia screen.

11. The surgical drape of claim 10, wherein the first armboard cover portion, second armboard cover portion, third armboard cover portion, and fourth armboard cover portion are further connected to the main body portion.

12. The surgical drape of claim 10, wherein the main body portion further includes:
a first outer main body portion extending towards the third side portion; and
a second outer main body portion extending towards the fourth side portion, wherein the first outer main body portion is connected to the first armboard cover portion and the second armboard cover portion, and the second outer main body portion is connected to the third armboard cover portion, and fourth armboard cover portion.

13. A method of forming a surgical drape, the surgical drape comprising:
assembling a main body portion with a fenestration for accessing a surgical site, the main body portion comprising:
a main body first side portion;
a main body second side portion;
a main body third side portion;
a main body fourth side portion; the surgical drape further comprising:
connecting a first anesthesia drape portion to extend away from the third side portion; and
connecting a second anesthesia drape portion to extend away from the fourth side portion.

14. The method of claim 13, wherein the first anesthesia drape portion and the second anesthesia drape portion are formed of a transparent material.

15. The method of claim 13, further comprising forming the fenestration closer to the first side portion than the second side portion.

16. The method of claim 14, further comprising providing a reinforcement portion at least partially surrounding the fenestration.

17. The method of claim 14, further comprising connecting a first outer body portion of the main body portion to the first anesthesia drape portion and connecting a second outer body portion to the second anesthesia drape portion.

18. A surgical drape system comprising:
a package containing an adhesive backed pocket and a surgical drape, the surgical drape comprising:
a main body portion with a fenestration for accessing a surgical site, the main body portion comprising:
a main body first side portion;
a main body second side portion opposite the first side portion;
a main body third side portion;
a main body fourth side portion opposite the third side portion; the surgical drape further comprising:
a first anesthesia screen extending away from the third side portion; and
a second anesthesia screen extending away from the fourth side portion.

19. The surgical drape system of claim 18, further comprising an adhesive portion, surrounding the fenestration.
